(19) United States
(12) Patent Application Publication
(10) Pub. No.: US 2026/0308505 A1
(43) Pub. Date: Oct. 8, 2026

(54) SAMPLING RETRACTOR SYSTEM AND METHOD OF USE

(71) Applicant: Cytiva US LLC, Marlborough, MA (US)

(72) Inventors: Ethan BARRIEAU, Marlborough, MA (US); James YOUNG, Marlborough, MA (US); Stuart JANG, Marlborough, MA (US); Nick DEROM, Marlborough, MA (US)

(73) Assignee: Cytiva US LLC, Marlborough, MA (US)

(21) Appl. No.: 19/485,538

(22) PCT Filed: Jun. 12, 2024
(86) PCT No.: PCT/US2024/033489

§ 371 Date: Nov. 18, 2025

Related U.S. Application Data
(60) Provisional application No. 63/521,385, filed on Jun. 16, 2023.
(60) Provisional application No. 63/521,389, filed on Jun. 16, 2023.

(51) Int. Cl. C12M 1/30; C12M 1/00; C12M 1/12; C12M 3/00

(52) U.S. Cl. CPC C12M 33/02; C12M 23/38; C12M 23/46; C12M 23/48; C12M 25/18

(57) ABSTRACT

A sampling device includes a rotatable knob, an end cap including a hollow connector, a hollow retraction chamber having a first chamber end and a second chamber end. Wherein the first chamber end is sealed against the rotatable knob, and the second chamber end is connected to, and in fluid communication with the end cap. A hollow tube having a tube first and tube second ends, the tube first end connected to, and in fluid communication with the hollow connector of the end cap, and the tube second end configured for connection to a lid of a fixed bed cell culture bioreactor system; a sampling element embedded inside the fixed-bed bioreactor system; and a retraction filament connecting the rotatable knob to the sampling element. Rotating the knob pulls the retraction filament and the sampling element upward through the hollow tube toward the rotatable knob and into the hollow retraction chamber.

### BACKGROUND OF THE INVENTION

Users of fixed-bed bioreactor systems may be required to collect and analyze a number of cell culture samples from the bioreactor systems. There is a need for improved sampling devices and methods of using them.

The present invention provides for ameliorating at least some of the disadvantages of the prior art. These and other advantages of the present invention will be apparent from the description as set forth below.

### BRIEF SUMMARY OF THE INVENTION

An aspect of the invention provides a sampling device for use in fixed-bed cell culture bioreactor systems comprising: A. a rotatable knob; B. an end cap including a hollow connector; C. a hollow retraction chamber having a first chamber end and a second chamber end, wherein the first chamber end is sealed against the rotatable knob, and the second chamber end is connected to, and in fluid communication with the end cap; D. a hollow tube having a tube first end and a tube second end, the tube first end connected to, and in fluid communication with the hollow connector of the end cap, and the tube second end configured for connection to a lid of a fixed-bed cell culture bioreactor system; E. a sampling element embedded inside the fixed-bed cell culture bioreactor system; and F. a retraction filament that connects the rotatable knob to the sampling element, wherein rotating the rotatable knob pulls the retraction filament and the sampling element in an upward direction through the hollow tube toward the rotatable knob and into the hollow retraction chamber.

The sampling element, in embodiments, is porous and comprises a porous medium, wherein the porous sampling element comprises a pleated porous sampling element comprising a pleated porous medium, the pleated porous medium being a continuous pleated porous medium.

The sampling element, in embodiments, comprises a plurality of arms.

The sampling device, in embodiments, further comprises a hollow insert cylinder interposed between the knob and the hollow retraction chamber.

In embodiments, rotating the rotatable knob pulls the retraction filament and the sampling element in an upward direction toward the rotatable knob and into the hollow retraction chamber until the sampling element is prevented from further upward movement and the retraction filament is broken, wherein the rotatable knob contains an aperture, wherein the retraction filament passes through the aperture, and a first stopper, attached to the filament, is seatable in the aperture to connect the retraction filament to the rotatable knob.

In embodiments, the sampling device further comprises a cassette, the cassette comprising: a frame having an open top end; a bottom end; a longitudinal axis from the open top end to the bottom end; a right side wall; a rear wall; a left side wall; a front wall; an open interior; and a horizontally arranged support between the top end and the bottom end; wherein the sampling element is located in the open interior of the frame, wherein the sampling element has an upper element end and a lower element end, and the aperture comprises horizontally arranged apertures passing through sampling element near the upper element end, the sampling element being supported at the lower element end by the horizontally arranged support, and wherein the retraction filament passes through the horizontally arranged apertures.

In embodiments, the retraction filament contains a second stopper near the sampling element; wherein the retraction filament passes through the aperture of the second stopper, through the aperture or horizontally arranged apertures of the sampling element, and further passes back through the stopper and forms a loop.

In embodiments, the open top end of the cassette frame has a retainer comprising at least two opposing inwardly facing legs, and the right side wall and the left side wall each have one of the at least two inwardly facing legs, and the front wall and the rear wall each have one of two opposing notches; or the rear wall and the front wall each have one of the at least two inwardly facing legs, and the right side wall and the left side wall each have one of the two opposing notches, and the sampling element is retained in the frame by the at least two inwardly facing legs.

In embodiments, the bottom end of the frame of the cassette further comprises two or more fingers configured to engage with a bottom plate in the fixed-bed cell culture bioreactor system.

Aspects of the invention provide a system use in a fixed-bed cell culture bioreactor system comprising a sampling device as described above, a second hollow connector connectable to the tube second end; a flange connectable to the second hollow connector; and a clamp configured to clamp the tube second end to the second hollow connector and the second hollow connector to the flange.

Aspects of the invention include a method for removing a sample from a fixed-bed bioreactor system and analyzing the removed sample, the method comprising rotating the rotatable knob of the sampling device to pull the retraction filament and the sampling element from a fixed bed cell culture bioreactor system into the hollow retraction chamber until the sampling element is prevented from further upward movement and the retraction filament is broken; and, while the hollow tube first end remains connected to, and in fluid communication with, the hollow connector of the retraction assembly, aseptically disconnecting the hollow tube from a lid of the fixed bed cell culture bioreactor system.

In embodiments, the retraction filament is broken when the second stopper prevents further movement of the filament during rotation of the rotatable knob.

### BRIEF DESCRIPTION OF THE SEVERAL VIEWS OF THE DRAWING(S)

FIGS. 1A to 1C are drawings showing an assembled sampling device according to aspects of the invention, including a rotatable knob, a retraction chamber, a hollow tube, and a sampling element attached to a retraction filament, wherein the sampling device is located in an illustrative fixed-bed bioreactor system comprising top and bottom plates and a lid. FIG. 1A shows the system in perspective view; FIG. 1B shows a transparent partial cut-away view of the system shown in FIG. 1A in first embodiment; FIG. 1C shows a transparent partial cut-away view of the system shown in FIG. 1A in a second embodiment.

FIG. 1D is a drawing showing an enlarged view of the lower portion shown in FIG. 1B, showing the sampling element located in the illustrative fixed-bed bioreactor system.

FIG. 1E shows an enlarged view of the lower portion shown in FIG. 1C, showing the cassette with the sampling element retained in the illustrative fixed-bed bioreactor system.

FIG. 1F is a drawing showing a transparent partial cut-away view of another aspect of an assembled sampling device according to an aspect of the invention located in the illustrative fixed-bed bioreactor system as shown in FIG. 1A.

FIG. 1G is a drawing showing an enlarged view of the lower portion shown in FIG. 1F, showing the sampling element located in the illustrative fixed-bed bioreactor system.

FIG. 1H is a drawing showing a transparent partial cut-away view of another aspect of an assembled sampling device according to an aspect of the invention located in the illustrative fixed-bed bioreactor system.

FIG. 1I is a drawing showing an enlarged view of the lower portion shown in FIG. 1F, showing the sampling element located in the illustrative fixed-bed bioreactor system.

FIGS. 2A and 2B are drawings showing, diagrammatically, the assembled sampling device shown in FIG. 1G, without showing the illustrative fixed-bed bioreactor system. FIGS. 2A and 2B show the assembled sampling device before rotation of the rotatable knob causes the sampling element to be withdrawn from the fixed-bed bioreactor system, wherein FIGS. 2A and 2B show, respectively, side and front views of the sampling element, and the top of FIG. 2A also includes a label showing the direction of knob rotation.

FIG. 2C is a drawing showing, diagrammatically, the assembled sampling device shown in FIG. 1H, without showing the illustrative fixed-bed bioreactor system.

FIGS. 2D and 2E are drawings showing, diagrammatically, the assembled sampling device shown in FIGS. 1A and 1C, without showing the illustrative fixed-bed bioreactor system. FIG. 2D shows the assembled sampling device before rotation of the rotatable knob, FIG. 2E shows the sampling element in the cassette, before rotation of the rotatable knob causes the sampling element to be withdrawn from the cassette, wherein the cassette will remain retained in the fixed-bed bioreactor system.

FIGS. 3A and 3B are drawings showing part of the sampling device (FIG. 3A) and the sampling device (FIG. 3B) shown FIGS. 2A and 2B, wherein the rotatable knob has been partially rotated, causing the sampling element to be withdrawn from the fixed-bed bioreactor system. FIG. 3B shows the sampling element withdrawn into the retraction chamber.

FIGS. 4A-4C are drawings showing the rotatable knob, FIG. 4A shows a perspective view; FIG. 4B shows a side view, and FIG. 4C shows a cross-sectional view along line A-A of FIG. 4B.

FIGS. 5A-5D are drawings, according to a first embodiment, showing aspects of the sampling element, wherein FIGS. 5A and 5B show, respectively, perspective and side views of an aspect of the sampling element showing the sampling element having single sheets of porous media, FIG. 5C shows a sampling element comprising pleated porous media, and FIG. 5D is a drawing showing another aspect of the sampling element with a plurality of porous media arms.

FIGS. 5E-5H are drawings, according to another embodiment, showing one aspect of the cassette for the sampling element; FIG. 5E shows a top perspective view with opposing notches at the top end, FIG. 5F shows top view, FIG. 5G shows a side view, FIG. 5H shows a partial cut-away view, also showing the sampling element attached to the retraction filament, and an optional perforated plate.

FIGS. 5I and 5J are drawings showing another aspect of the cassette for the sampling element of FIGS. 5E-5H, FIG. 5I shows a top perspective view, showing opposing notches, optional opposing fingers, and optional retainers for attachment to a bioreactor system; FIG. 5J shows a side view.

FIG. 5K is a drawing showing an aspect of the sampling element of FIGS. 5E-5H.

FIGS. 6A-6B are drawings showing an insert chamber that is inserted in the retraction chamber, the insert chamber receiving the rotatable knob. FIG. 6A shows a top perspective view, FIG. 6B shows a front side view.

FIGS. 7A-7E are drawings showing an aspect of the retraction chamber. FIG. 7A shows a top perspective view, FIG. 7B shows a top view, FIG. 7C shows a cross-sectional view along line 7C-7C of FIG. 7B; FIG. 7D shows a front side view, FIG. 7E shows a different side view.

FIGS. 8A-8E are drawings showing the rotatable knob (retraction filament not shown), some drawings showing the knob passing through the insert chamber, wherein the insert chamber is engaged with the retraction chamber at one end of the retraction chamber, and a threaded end cap including a hollow connector engaged with the retraction chamber at the other end of the retraction chamber. FIG. 8A is a top perspective view; FIG. 8B shows a top view, FIG. 8C shows a cross-sectional view along line 8C-8C of FIG. 8B.

FIGS. 8D and 8E are drawings showing views similar to those shown in FIGS. 8A and 8C, for use with smaller samples than those used with the aspect shown in FIGS. 8A and 8C, wherein the knob, insert chamber, and threaded end cap including a hollow connector are reduced in size compared to those in FIGS. 8A and 8C.

FIG. 9 is a drawing showing a side view of an aspect of an assembled retraction assembly, comprising the retraction filament (attached first stopper not shown) and attached second stopper, also showing the aspects of the rotatable knob, the insert chamber, the retraction chamber and the threaded end cap including a hollow connector shown in FIGS. 8A-8E, wherein the rotatable knob has been rotated such that the retraction filament has pulled the sampling element out of the fixed-bed bioreactor system into the retraction chamber. FIG. 9 also shows tamper evident seals allowing the user to determine if retraction assembly has been tampered with and/or prematurely opened.

FIGS. 10A-10D are drawing illustrating an embodiment for locking the endcap after the filament break.

FIGS. 10E and 10F are drawings illustrating an alternative embodiment for locking the endcap after the filament break.

FIG. 11 is a drawing showing a perspective view of the threaded end cap including a first hollow connector.

FIGS. 12A and 12B are drawings showing perspective views of second hollow connectors for connecting to the hollow tube second end to a sanitary flange, as shown respectively in FIGS. 1B and 1H.

FIG. 13 is a drawing showing a perspective view of the sanitary flange for connecting the second hollow connector to the lid of a fixed-bed cell culture bioreactor system (clamp for maintaining the connection between the second hollow connector and the sanitary flange not shown).

FIGS. 14A-14B are, respectively, drawings of a top view and a bottom perspective view of an illustrative clamp (shown as a tri-clover clamp) for maintaining the connection between the second hollow connector and the sanitary flange

### DETAILED DESCRIPTION OF THE INVENTION

In accordance with an aspect of the invention, a sampling device for use in fixed-bed cell culture bioreactor systems is provided, comprising: A. a retraction assembly comprising: (a) a retraction filament having an attached first stopper and an attached second stopper; (b) a rotatable knob connected to the retraction filament, the rotatable knob having an aperture, wherein the retraction filament passes through the aperture, and the attached first stopper is seatable in the aperture and the attached second stopper is spaced a distance from the attached first stopper; (c) a threaded end cap including a hollow connector; (d) a hollow retraction chamber having a first chamber end and a second chamber end, wherein the first chamber end is sealed against the rotatable knob, and the second chamber end is threadably connected to, and in fluid communication with, the threaded end cap, wherein the retraction filament is also connected to a porous sampling element, and rotating the rotatable knob pulls the retraction filament and the porous sampling element in an upward direction toward the rotatable knob and into the hollow retraction chamber until the attached second stopper is prevented from further upward movement and the retraction filament is broken; B. a hollow tube having a tube first end and a tube second end, the tube first end connected to, and in fluid communication with the hollow connector of the retraction assembly, and the tube second end configured for connection to a lid of a fixed-bed cell culture bioreactor system, wherein the retraction filament passes from the rotatable knob and through the tube second end; and C. a porous sampling element comprising a porous medium, wherein the porous sampling element has an aperture passing through the porous sampling element; wherein the retraction filament passes through the aperture, the retraction filament forming a loop including the attached second stopper.

In an aspect of the sampling device, the porous sampling element has an upper element end and a lower element end, an aperture passing through the porous sampling element near the upper element end; wherein the retraction filament passes through the aperture, the retraction filament forming a loop including the attached second stopper.

In accordance with an aspect of the invention, a method for removing a cell sample from a fixed-bed bioreactor system comprises rotating the rotatable knob of an aspect of the retraction assembly of the sampling device to pull the retraction filament, attached second stopper, and the porous sampling element from a fixed-bed cell culture bioreactor system into the hollow retraction chamber until the attached second stopper is prevented from further upward movement and the retraction filament is broken; and, while the hollow tube first end remains connected to, and in fluid communication with, the hollow connector of the retraction assembly, aseptically disconnecting the hollow tube from a lid of the fixed-bed cell culture bioreactor system. Preferably, the method includes removing a tamper evident seal from the knob before rotating the knob.

In accordance with another aspect of the invention, a method of analyzing a sample from a fixed-bed bioreactor system comprises removing the threaded end cap of an aspect of the sampling device from a second chamber end of a hollow retraction chamber containing a porous sampling element removed from a fixed-bed bioreactor system, and analyzing the sample adhered to the porous sampling element. Preferably, the hollow retraction chamber containing the removed porous sampling element is placed in a biosafety cabinet before removing the threaded end cap from the second chamber end of the hollow retraction chamber, e.g., to avoid damaging and/or contaminating the sample and/or exposing the user to a health hazard. In some aspects, the hollow retraction chamber has at least one tamper evident seal that is removed after placement of the hollow retraction chamber in the biosafety cabinet, before removing the threaded end cap from the second chamber end.

Typically, analyzing the sample comprises determining the cell population density of the cells adhered to the sampling element. Alternatively, or additionally, analyzing the sample comprises one or more of replication competent retroviral testing, adventitious virus testing, and sterility testing (e.g., wherein the sampling element with adhered cells is placed in liquid media to analyze for exogenous microbial growth).

Advantageously, aspects of the device and method of use allow for sterile/aseptic removal and analysis of a sample from the bioreactor system without damaging and/or contaminating the sample and/or the bioreactor system, or exposing the operator to a health hazard associated with the sample, such as a virus. Preferably, the sampling device has a configuration for alerting the user (e.g., audibly and/or by feeling the “snap” of engagement and/or by less resistance to knob rotation) of the completion of the sampling action. The device preferably has seals to prevent the ingress of contaminating particles.

Another aspect of the invention comprises a system for use in a fixed-bed cell culture bioreactor system comprising an aspect of the sampling device; a second hollow connector connectable to the tube second end; a flange connectable to the second hollow connector; and, a clamp configured to clamp the tube second end to the second hollow connector and the second hollow connector to the flange.

Each of the components of the invention will now be described in more detail below, wherein like components have like reference numbers.

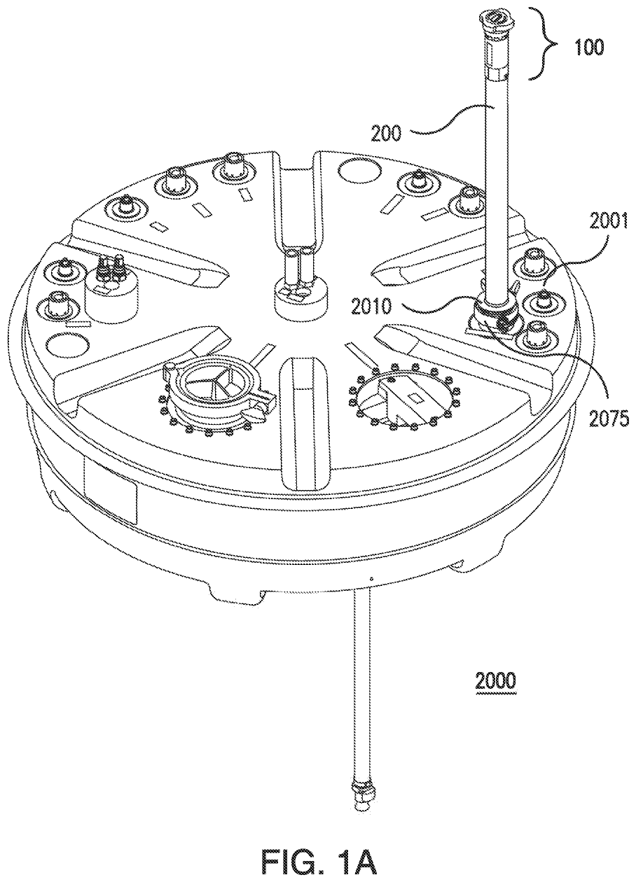

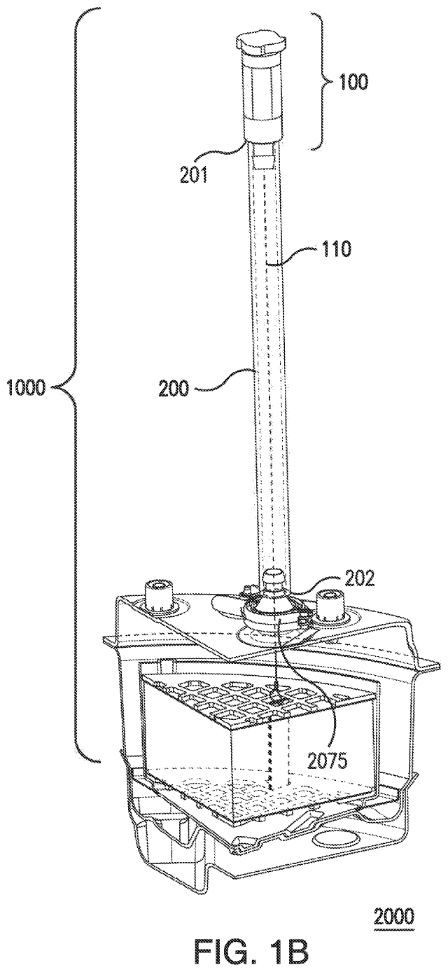

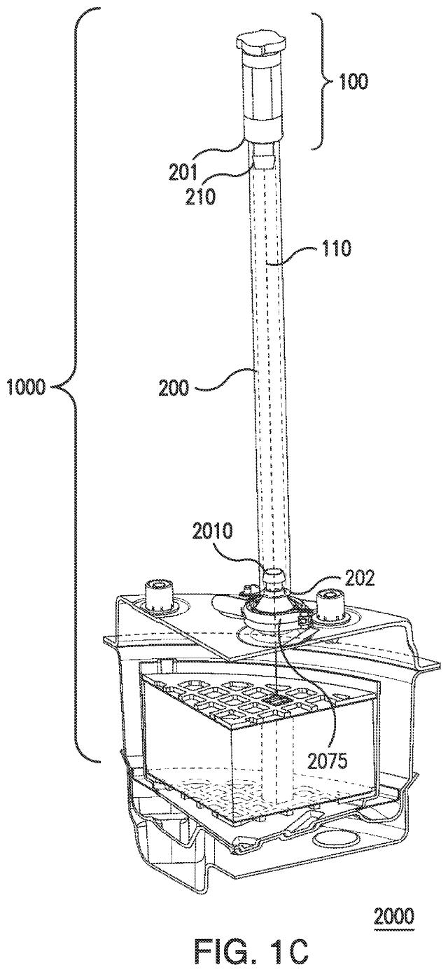

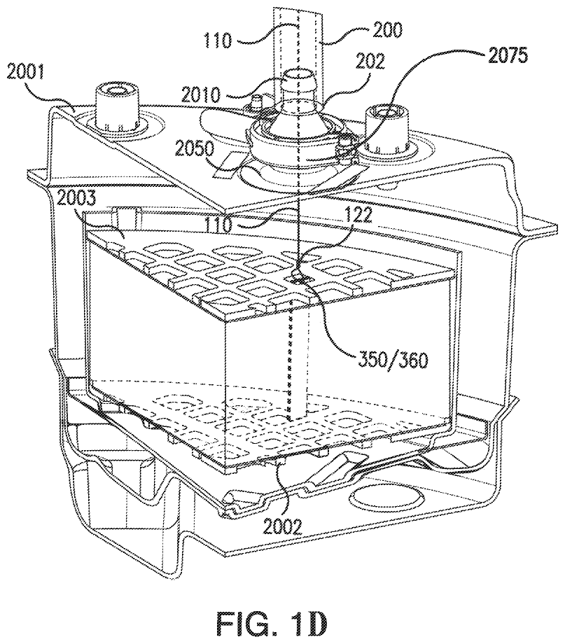

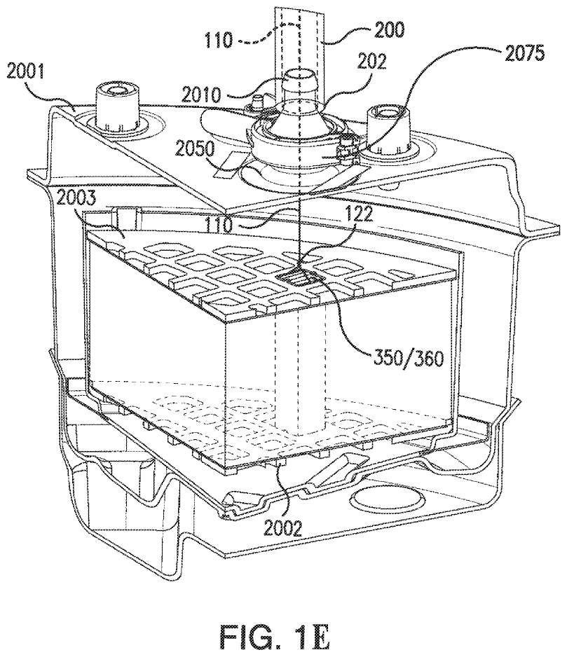

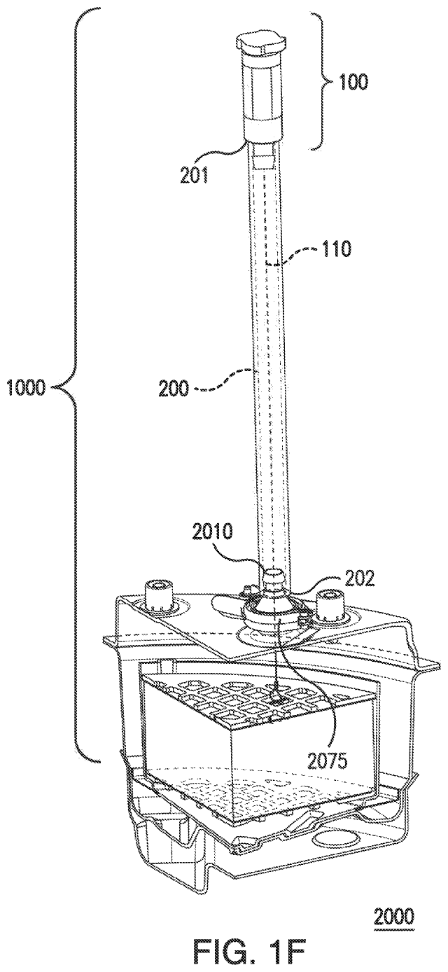

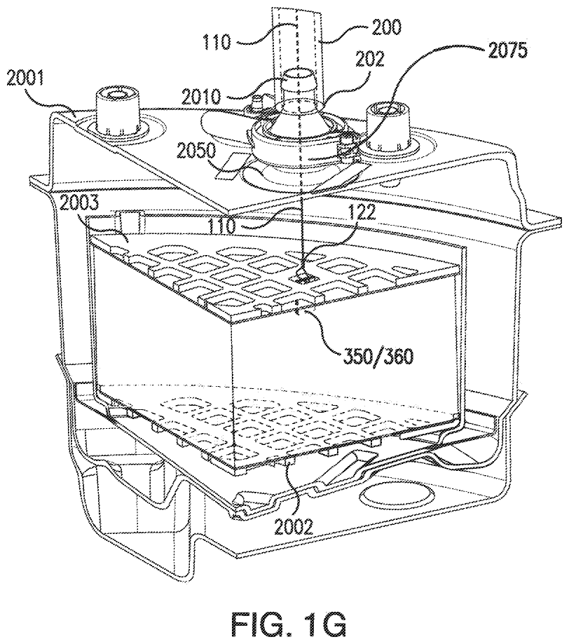

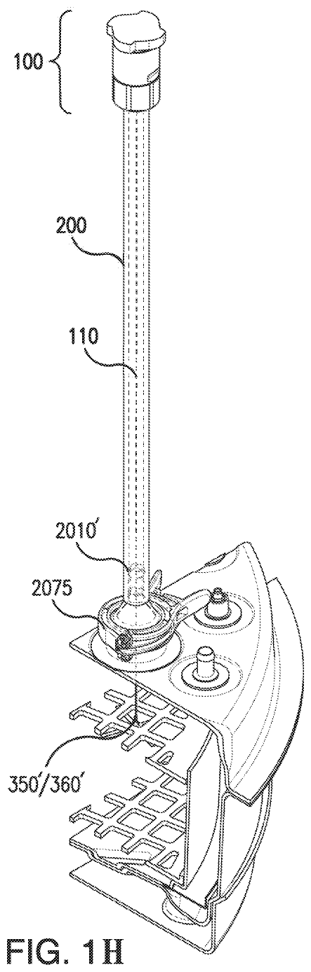

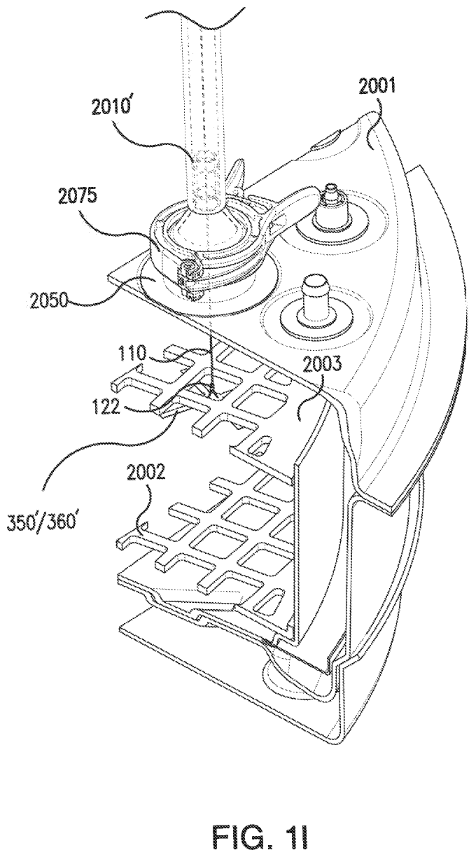

Using the illustrated aspects shown in FIGS. 1A-1I for reference, the sampling device 1000 for use in fixed-bed cell culture bioreactor systems comprises a retraction assembly 100; a hollow tube 200; and a sampling element 350 (FIGS. 1B, 1D, 1F-1G), a sampling element 350′ (FIG. 1H and 1i), or a cassette 300 (FIGS. 1C and 1E), as discussed in more detail below.

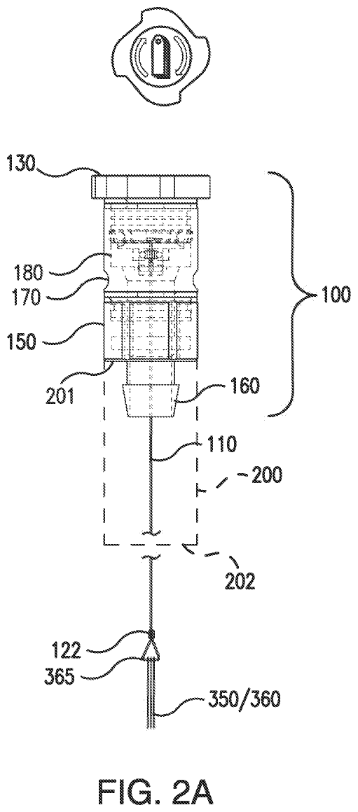

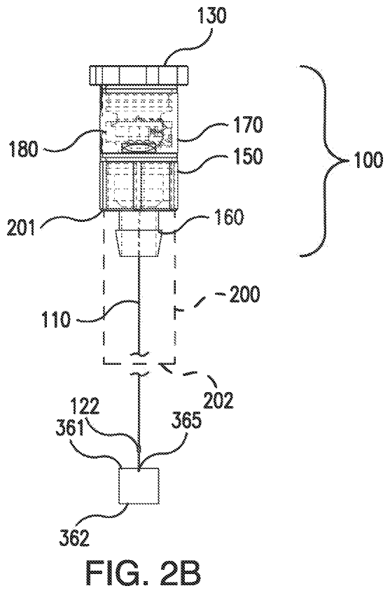

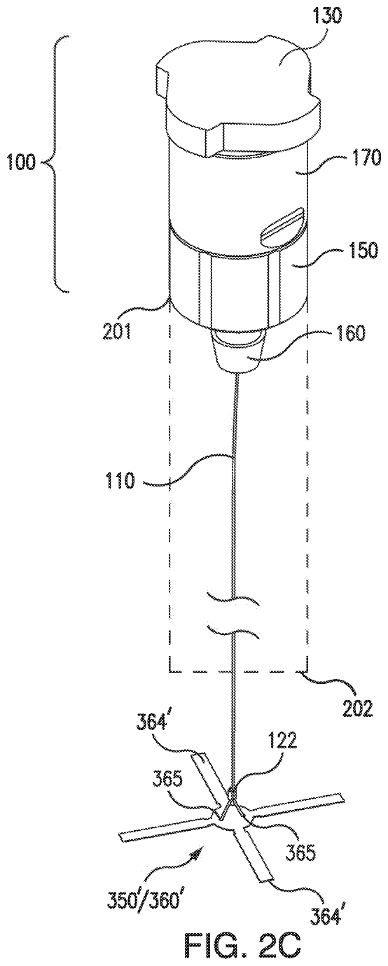

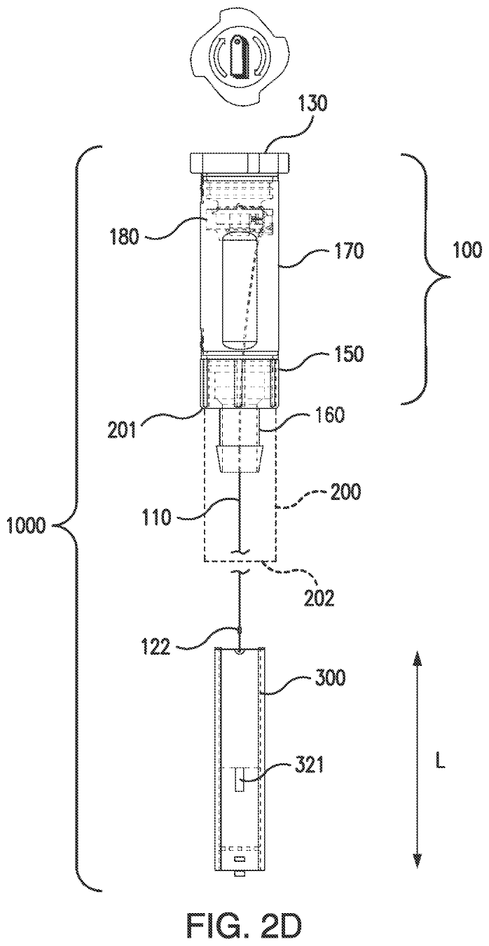

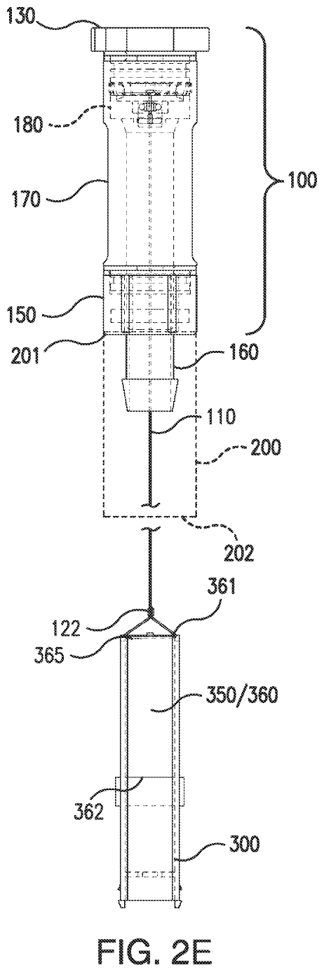

The illustrated retraction assembly 100 comprises (a) a retraction filament 110 having an attached first stopper 121 and attached second stopper 122 (see, for example, FIGS. 2A-2E, 3A-3B, and 5D);

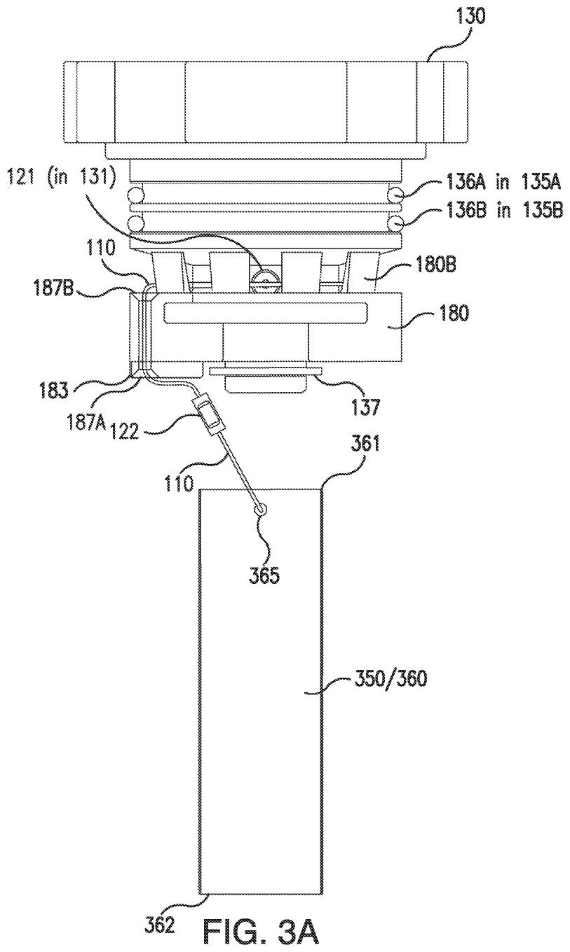

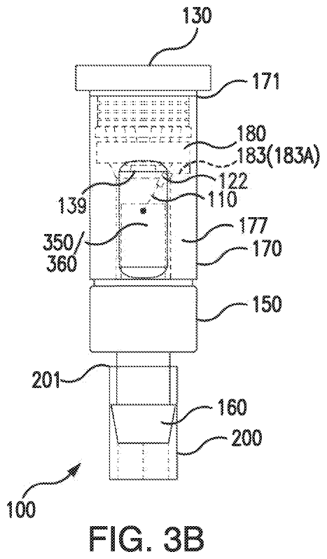

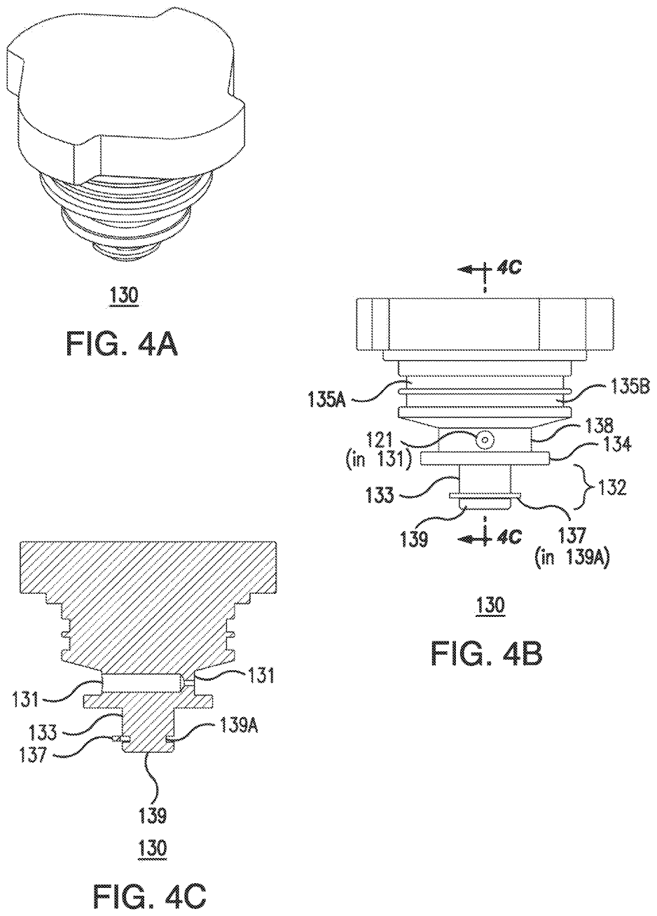

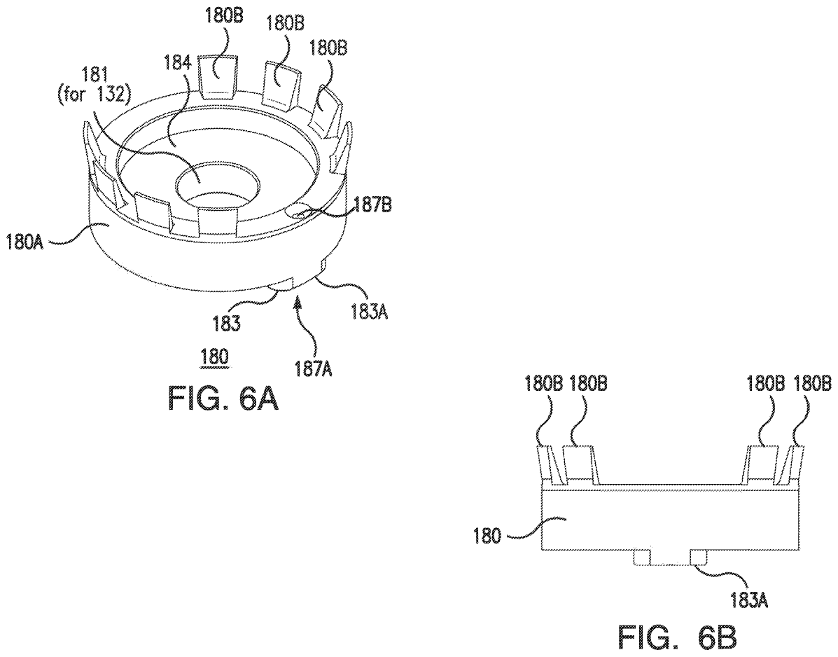

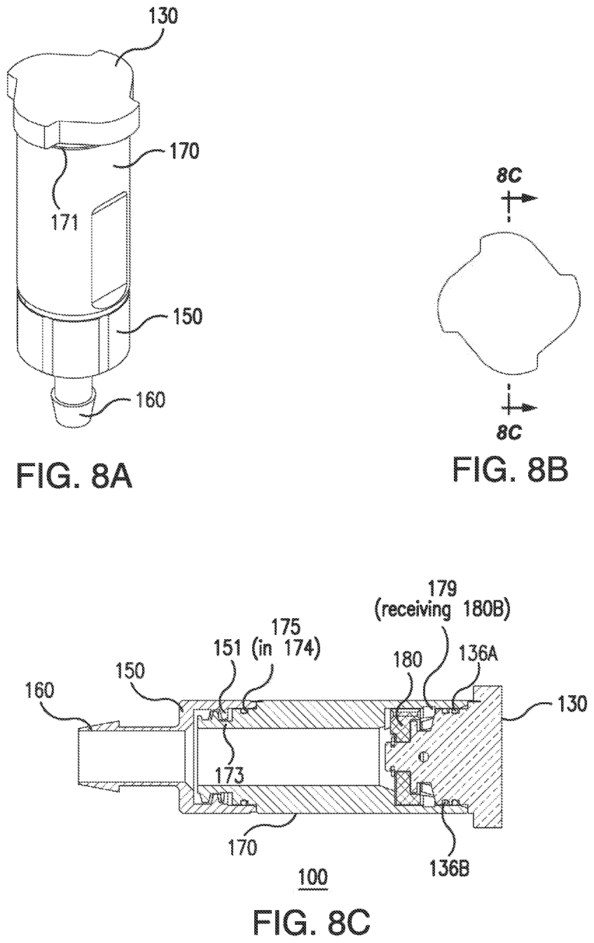

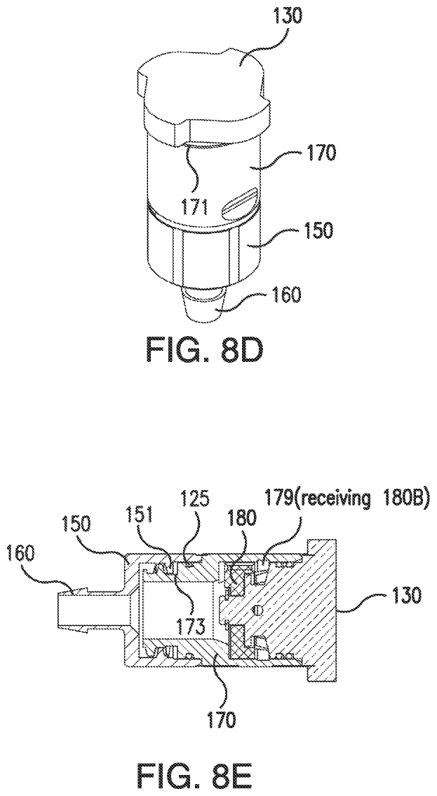

(b) a rotatable knob 130 (the illustrated knob has four curved protrusions for ease of gripping by the hand of the operator) connected to the retraction filament, the rotatable knob having an aperture 131 (see, for example, FIG. 3A, and 4B-4C; the aperture preferably passes from one side of the knob through the other side) located in a knob side wall portion forming a groove 138 (for receiving the wound retraction filament, see, FIG. 4B), wherein the retraction filament passes through the aperture, and the attached stopper is seated in the aperture, the knob also having a base 134, a knob stem 132 having an outer surface 133, a tip 139, and a groove 139A in the stem (wherein the groove 139A receives a retaining ring 137 (such as a c-clip as shown in FIG. 4C) for retaining an insert cylinder 180 (see, FIGS. 6A-6B, 8C, and 8E); an upper portion of the knob having grooves 135A and 135B for receiving resilient members 136A, 136B such as o-rings (see, FIGS. 3A, 4B, and 8C);

(c) a threaded end cap 150 including internal threads 151 and a first hollow connector 160 (shown as a hose barb) (see, for example, FIGS. 2A-2C, 8C, 8E, and 11);

(d) a hollow retraction chamber 170 having a first chamber end 171 and a second chamber end 172, a base 176, a central passage 177, and an inner surface with a snap groove 179 near the first end 171 (see, FIGS. 2A, 3B, 7A-7D), wherein the first chamber end is sealed against the rotatable knob (see, FIGS. 3A, 8C, and 8E, wherein resilient members 136A, 136B seal against the inner surface of the first chamber end), and the second chamber end is threadably connected to, and in fluid communication with, the threaded end cap (the second chamber end 172 having an outer surface with threads 173 and a groove 174 (for receiving a resilient member 175 such as an o-ring), the threaded end cap 150 having an inner surface 151 with threads (see, FIGS. 8C and 8E); wherein the retraction filament 110 is also connected to a porous sampling element 350 (see, FIGS. 1D, 1G, 2A-2B), porous sampling element 350′ (see, FIGS. 1I and 2C), or a pleated porous sampling element 350 (arranged in a cassette 300) (see, FIGS. 1E, 2D-2E), and rotating the rotatable knob pulls the retraction filament and the porous sampling element from the fixed-bed bioreactor system in an upward direction toward the rotatable knob and into the hollow retraction chamber 170, winding the retraction element around the knob until the first stopper 121 contacts the aperture 131, subsequently continuing to rotate the knob until the second stopper 122 contacts the shoulder 183 and surface 183A and the retraction filament 110 breaks, preventing further upward movement of the retraction filament (see, FIG. 3B).

In alternative embodiments, the threads are omitted and another mechanism for connecting elements of the sampling device 1000 in employed. For example, a bayonet style connection, a tab and recess connection, a snap fit, or a magnetic connection can be employed to connect various components of the sampling device 1000 (e.g., connection of the end cap 150 to retraction chamber 170, knob 130 to retraction chamber 170, etc.).

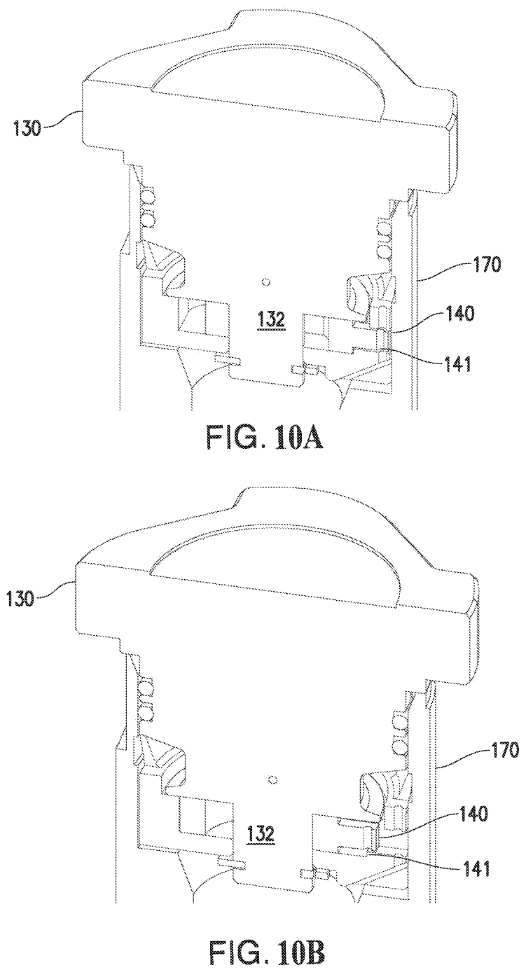

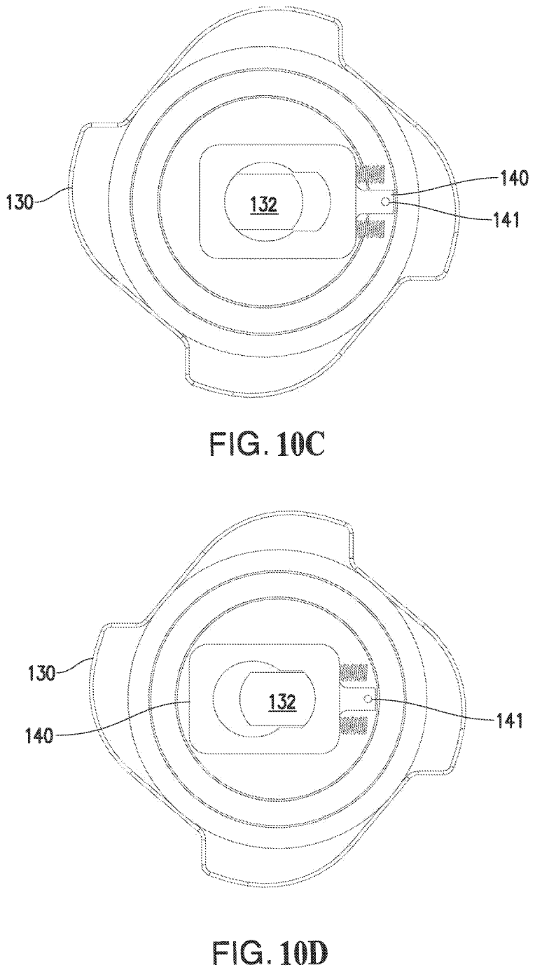

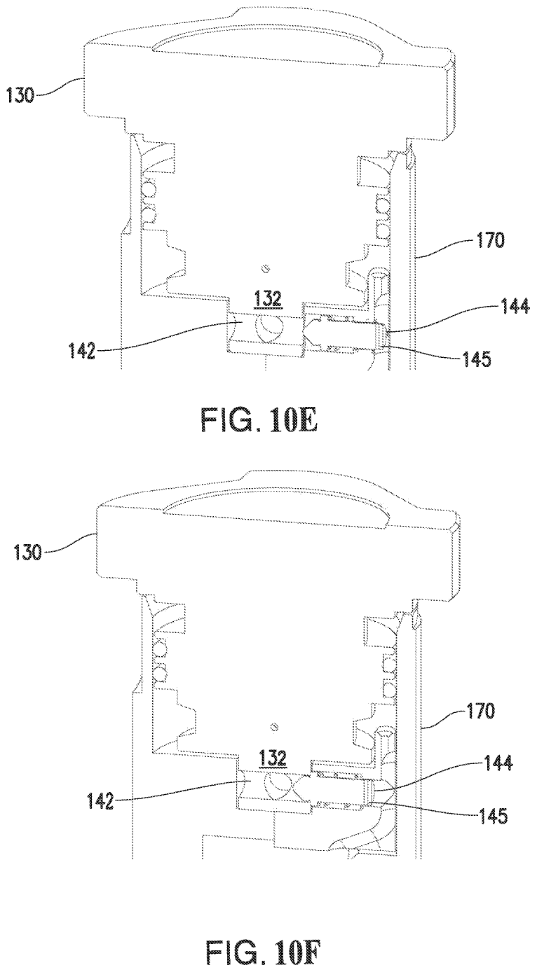

In embodiments, the retraction chamber 170 may include a locking mechanism 140, 142 (see, FIGS. 10A-10F, which is configured to prevent further rotation of knob 130 when the retraction filament 110 breaks. In one embodiment, as depicted in FIGS. 10A-10D, locking mechanism 140 takes the form of a spring-loaded stopper that is configured to engage knob stem 132. As best shown in FIGS. 10C and 10D, locking mechanism 140 includes an aperture 141 that the retraction filament 110 is fed through. Prior to retraction, the filament 110 prevents the locking mechanism 140 from engaging with the knob stem 132 (FIG. 10C). Once the filament 110 is broken, the springs force the locking mechanism to engage the knob stem 132 (FIG. 10D) and is shaped such that the stem cannot be rotated further. In a further embodiment, as depicted in FIGS. 10E and 10F, locking mechanism 142 is a spring-loaded stopper that is configured to engage an through hole in knob stem 132. Similar to the previous embodiment, locking mechanism 142 includes an aperture 145 though which filament 110 is fed. Prior to retraction, the filament 110 prevents the locking mechanism 140 from engaging with the knob stem 132 (FIG. 10E). ). Once the filament 110 is broken, the springs force the locking mechanism 142 to engage the through hole in knob stem 132 (FIG. 10F), thereby preventing further rotation of knob 130.

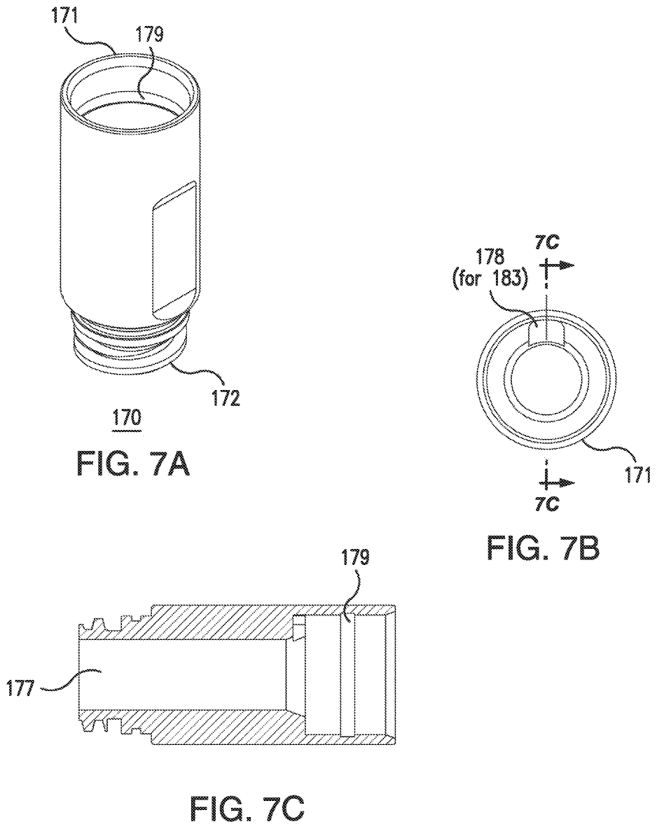

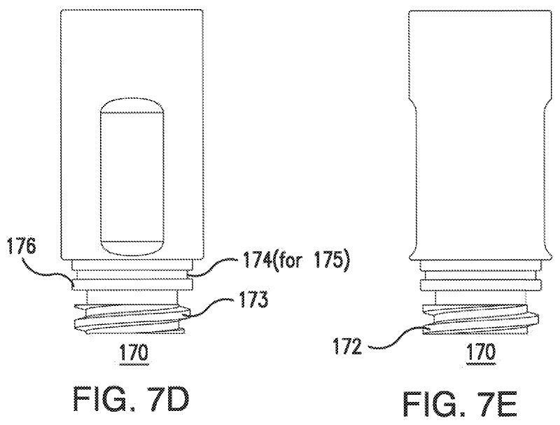

In the illustrated aspect of the hollow retraction chamber shown in FIGS. 7A-7D, the inner surface has an inwardly facing projection a distance below the snap groove 179, the projection extending toward the second end 172, the projection having a cut-out 178 at a top end of the projection below the snap groove, the cut-out for receiving a shoulder 183 of an insert cylinder 180 (see, FIGS. 6A-6B), and the snap groove 179 for receiving arms 180B of the insert cylinder (discussed below).

In the aspect as shown in FIGS. 6A-6B, 8C and 8D, the retraction assembly includes a hollow insert cylinder 180 interposed between the knob and the hollow retraction chamber, the insert cylinder having a side wall 180A with a plurality of upwardly facing (facing away from second end 172) outwardly angled arms 180B (that can snap fit into the snap groove 179 of the retraction chamber 170, see, FIGS. 7A, 7C, 8C and 8E) and a hole 181 (for receiving the knob stem 132) surrounded by a recessed surface 184 (the lower surface of the base 134 of the knob contacting the recessed surface 184), and (on a lower side facing the first chamber end 171) an extending shoulder 183 for receiving (through openings 187A and 187B) the retraction filament 110 (the shoulder having a surface 183A facing the direction of the second chamber end 172; wherein the shoulder 183 and surface 183A can be received into cut-out 178, see, FIG. 7B).

The illustrated hollow tube 200 shown in FIGS. 1A-1B, and 2A-2C, has a tube first end 201 and a tube second end 202, the tube first end connected to, and in fluid communication with the first hollow connector 160 of the retraction assembly, and the tube second end 201 configured for connection to a lid 2001 of a fixed-bed cell culture bioreactor system 2000 (e.g., using a sanitary flange 2050, a connector 2010 or 2010′, and a clamp 2075; see, FIGS. 1D, 1G, and 1I), wherein the retraction filament passes 110 from the rotatable knob and through the tube second end 202.

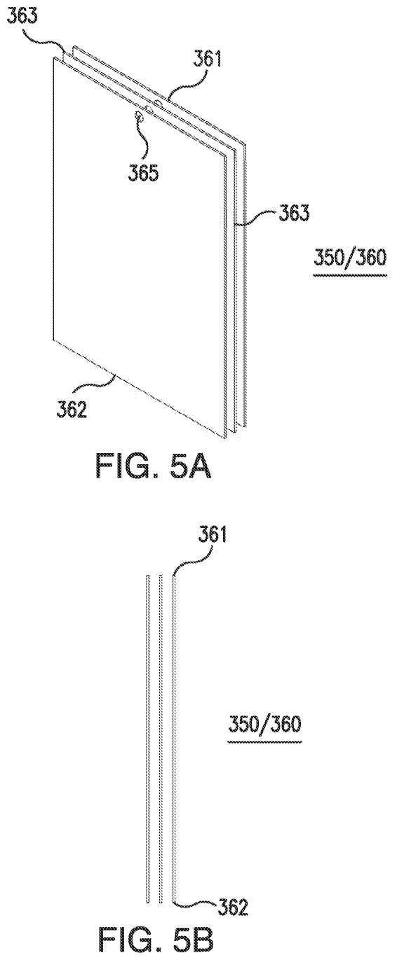

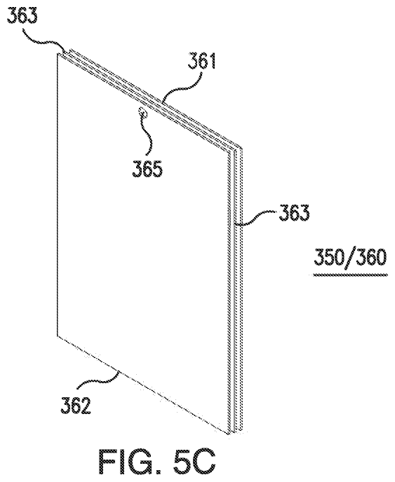

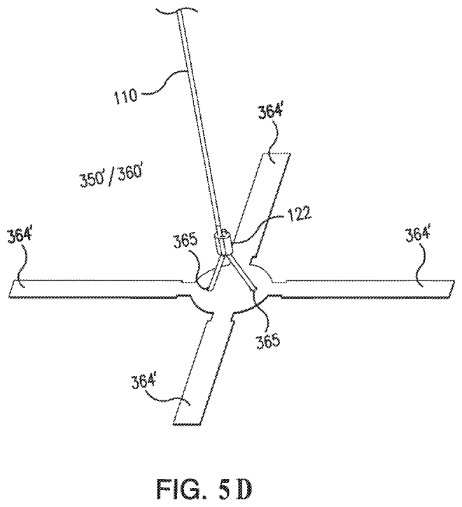

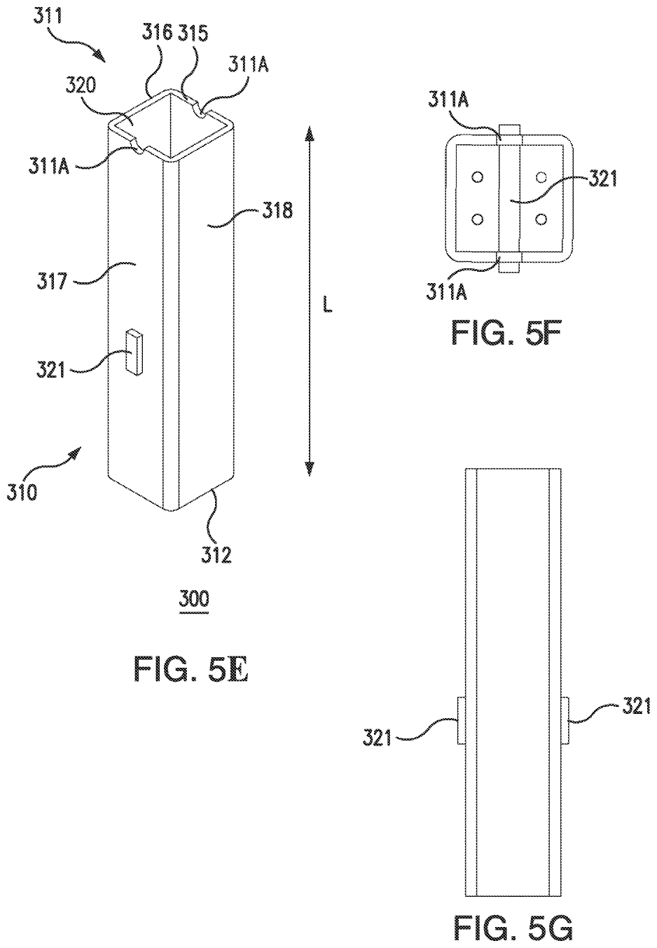

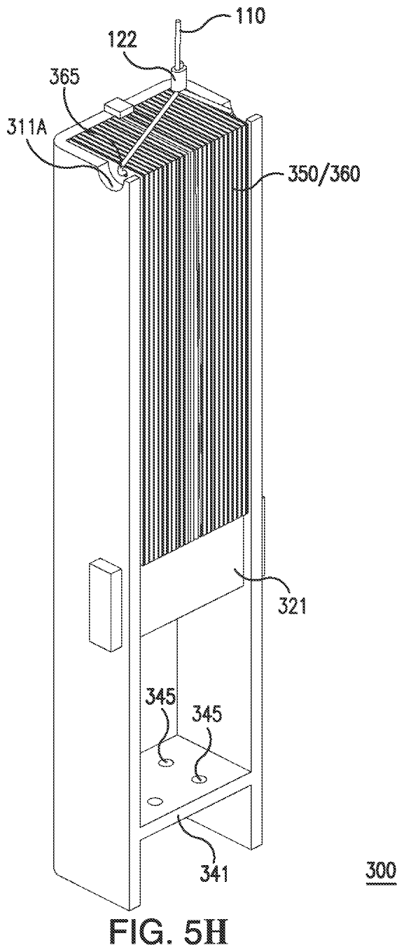

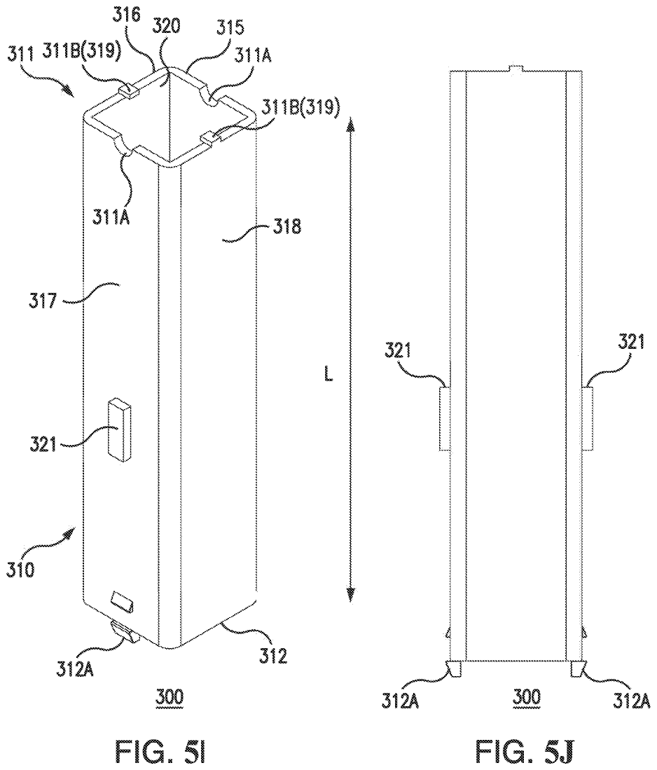

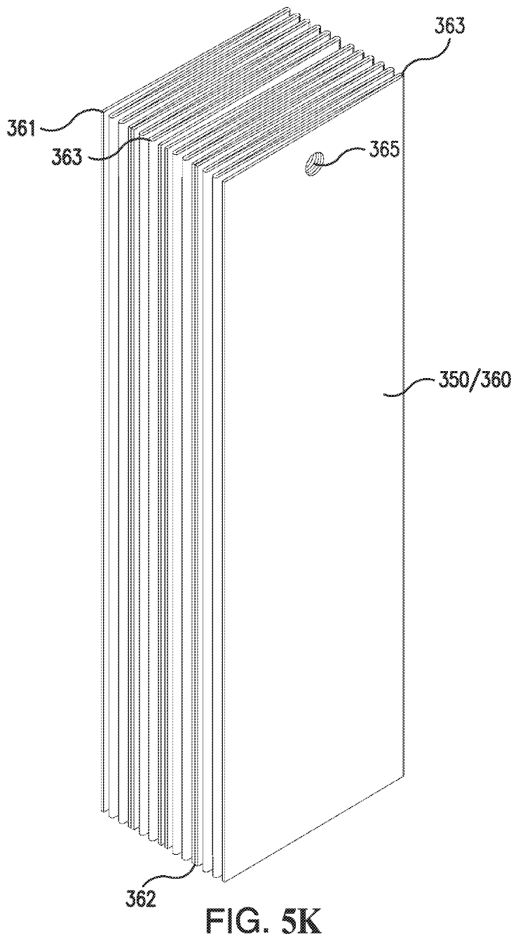

In embodiments. the porous sampling element 350 comprising at least one porous medium 360 (3 sheets or porous media are shown in FIGS. 5A-5B, in some aspects, as shown in FIG. 5C, the porous sampling element comprises a porous pleated sampling element comprising a porous pleated medium having pleats 363) arranged in the open interior of the frame, wherein the porous sampling element has an upper element end 361 and a lower element end 362, and an aperture 365 passing through the porous sampling element near the upper element end (see, FIGS. 3A, 5A-5C, wherein FIG. 5C shows a plurality of apertures, an aperture passing through sheet/porous medium). In another aspect, as shown in FIGS. 2C and 5D, the porous sampling element 350′ comprising at least one porous medium 360′, the sampling element 350′/porous medium 360′ comprising a plurality of arms 364′ (4 arms are shown, aspects can include any number of arms.

In further embodiments, a cassette 300 illustrated in FIGS. 5E-5J comprise (e) a frame 310 having an open top end 311 comprising at least two opposing notches 311A (the frame optionally also having optional retainer 319 illustrated as two opposing inwardly facing legs 311B as shown in FIGS. 51-5J); a bottom end 312; a longitudinal axis L from the open top end to the bottom end; a right side wall 315; a rear wall 316; a left side wall 317; a front wall 318; an open interior 320; and a horizontally arranged support 321 between the top end and the bottom end, wherein either the right side wall and the left side wall each have one of the at least two opposing notches at the open top end, or the rear wall and the front wall each have one of the two opposing notches at the open top end; in those aspects also having two opposing inwardly facing legs, the opposing walls not including the opposing notches include the opposing inwardly facing legs. While the pleated porous sampling element can be frictionally retained in the cassette by contact with the inner surfaces of the walls without inwardly facing legs, the legs can assist in retaining the element in the cassette.

The cassette also includes (f) a pleated porous sampling element 350 comprising a porous pleated medium 360 arranged in the open interior of the frame, wherein the pleated porous sampling element has an upper element end 361, a lower element end 362, and a plurality of pleats 363, and horizontally arranged apertures 365 passing through the pleated porous sampling element near the upper element end (see, FIGS. 3A, 5H, 5K), wherein the retraction filament passes through the horizontally arranged apertures, the pleated porous sampling element optionally being additionally retained in the frame 310 by the at least two inwardly facing legs 311B (of the aspect of the retainer as shown in FIGS. 5I and 5J) and being supported at the lower element end by the horizontally arranged support 321; wherein the retraction filament passes through the two opposing notches in the frame, and connected as a loop by a crimped attached second stopper 122, wherein the retraction filament 110 will be broken when the attached second stopper 122 is prevented from further upward movement upon contact with surface 183A of the insert cylinder 180 (see, FIGS. 3A and 3B) as the knob is further rotated.

In some aspects, the cassette 300 has an optional perforated horizontal plate 341 (see, FIG. 5H) arranged between the horizontally arranged support 321 and the bottom end 312 of the cassette, the perforated horizontal place having adjustable-size flow restriction holes 345 to balance the flow through the sampling element relative to flow through the fixed bed. In some aspects, the horizontal plate has in the range of 3 to 6 perforations (restriction holes 345) each hole having a diameter in the range of 1 to 6 mm in diameter.

In some aspects, the bottom end 312 includes at least two fingers 312A (see, FIGS. 5I-5J), wherein either the right side wall and the left side wall at the bottom end each have one of the at least two fingers; or, either the rear wall and the front side wall at the bottom each have one of the at least two fingers, for engagement with the bottom plate 2002 of the bioreactor system (see, FIG. 1E). In other aspects (e.g., using FIG. 1E for reference), fingers are not present, e.g., the cassette can retained between the top and bottom plates 2003, 2002 of the reactor system as the bioreactor system is being assembled and/or the outer surfaces of the walls at the bottom end can be frictionally engaged (e.g., press-fit) with openings in the bottom plates.

As shown in, for example, FIGS. 3A and 3B, regardless of whether the cassette frame is retained by or engaged with the reactor system, the pleated porous sampling element is pulled from the cassette frame 310 by the retraction filament into the retraction chamber 170 (see, FIG. 3B) as the retraction filament is pulled toward the knob.

In all of these aspects of the porous sampling element 350 350′, the retraction filament passes through the aperture(s), wherein the retraction filament is connected as a loop by a crimped attached second stopper 122, wherein the retraction filament 110 will be broken when the attached second stopper 122 is prevented from further upward movement upon contact with surface 183A of the insert cylinder 180 (see, FIGS. 3A and 3B) as the knob is further rotated.

As shown in, for example, FIGS. 3A and 3B, the porous sampling element is pulled by the retraction filament into the retraction chamber 170 (see, FIG. 3B) as the retraction filament is pulled toward the knob. In those aspects wherein the porous sampling element has a plurality of arms (e.g., 364′ in FIG. 5D), the arms can bend, e.g., downwardly, as the retraction filament is pulled toward the knob.

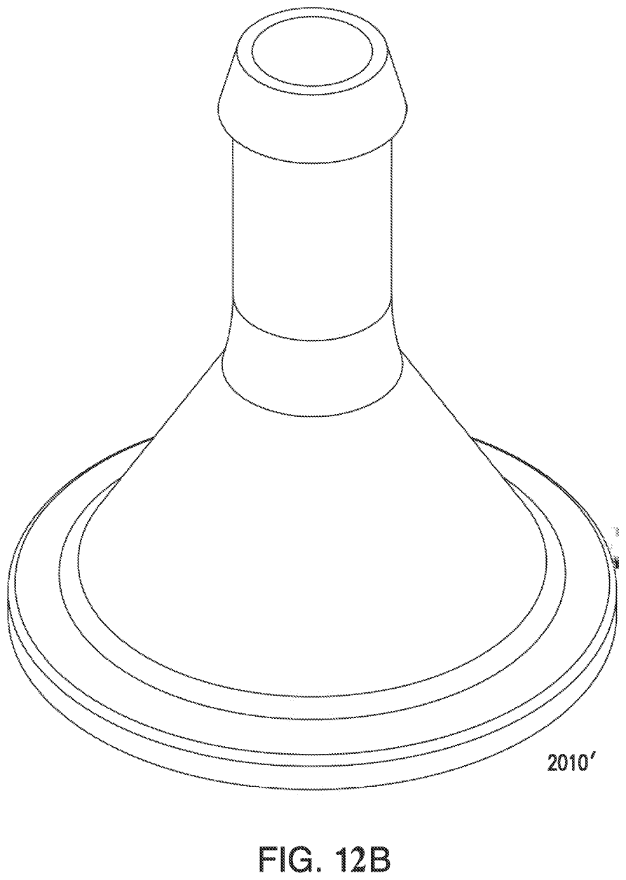

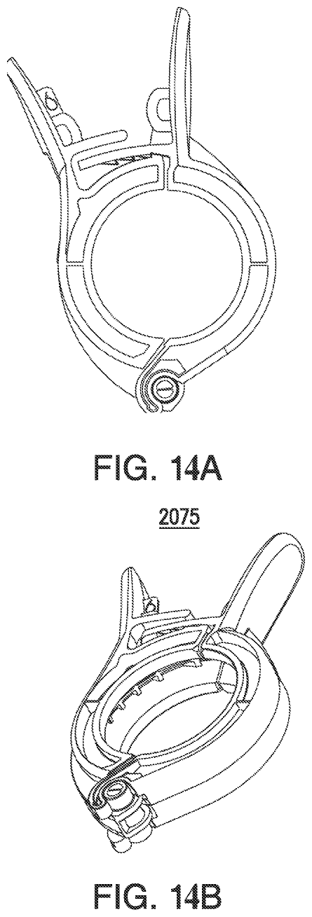

The sampling device is connected to the fixed-bed culture system 2000, preferably using a sanitary flange 2050, a second hollow connector 2010 or 2010′ (shown as hose barbs) and a clamp 2075 as shown in FIGS. 1A-1I and 14A-14B, see also, FIGS. 12A, 12B, and 12, wherein the second end 202 of the hollow tube 200 is connected to the connector 2010, or 2010′, the connector is connected to the sanitary flange 2050, and the clamp 2075 forms a seal between the tube, the connector, and the flange.

The sampling element, which can be a comprise a single sheet of porous media, typically a plurality of separate sheets of porous media, a single sheet of porous media comprising a plurality of arms (see, FIGS. 5A, 5B, and 5D), or can comprise a continuous pleated porous medium, or can comprise a combination of separate pleats and at least one continuous pleated medium (see, FIG. 5C),. In those aspects wherein the sampling element comprises a pleated element, the element can have any number of pleats, for example, in the range of 1 to 100 pleats, typically 10 to 75 pleats. For FDA replication competent retroviral testing, the sampling element can have, for example 25 to 30 pleats.

The sampling element can have any suitable dimensions (see, for example, the sampling elements illustrated in FIGS. 1C, 1D, 2B, 2E, and 5D (e.g., with respect to each arm 364′)), typically a length in the range of 2 cm and 10 cm, and a width in the range of 6 mm to 15 mm.

In an aspect implementing the cassette 300, the sampling element takes up in the range of from 40% to 85% of flow area in the cassette, more preferably, in the range of from 50% to 70% of the flow area, to create flow channels which promote homogenous cell distribution in and on the element during seeding and provide adequate flow of cell culture fluid to support cell growth. The pleat density is independent of the height, and so the height can be scaled based on the design and specific needs of the bioreactor. Illustratively, if the cassette has a fixed internal cavity width of 40 mm, 66 pleats would equal 76% pleat density. 100% pleat density is when the combined thicknesses of all of the pleats inside the cassette equals the width of the internal cavity of the cassette frame (e.g., if the width of the internal cavity is 40 mm, and the thickness per leg of the pleat is 0.23 mm, or 0.46 mm per pleat, 40 mm/0.46 mm=87 pleats for 100% pleat density). In this configuration, the entire cassette is filled with porous media and no space is available in between for cells or cell culture media to flow. To allow for flow channels, the number of pleats is reduced to less than 100% of the theoretical maximal amount of pleats that fit in the cassette frame.

In accordance with aspects of the invention, the sampling element, which is hydrophilic, preferably flexible, and formed from the same material used as the fixed bed, can be a woven or a non-woven porous medium, or a porous membrane, and can be formed from any of numerous materials, including those known in the art, including, for example, a natural or, more preferably, a synthetic polymer, such as polyethylene terephthalate (PET).

One example of a suitable non-woven porous medium is non-woven hydrophilized polyethylene terephthalate (PET).

The retraction filament typically has an ultimate tensile strength in the range of 2.5 pounds force (lbf) to 100 lbf. Suitable materials for the filament are biocompatible materials such as, for example, titanium, nichrome, stainless steel, polyethylene, and polypropylene.

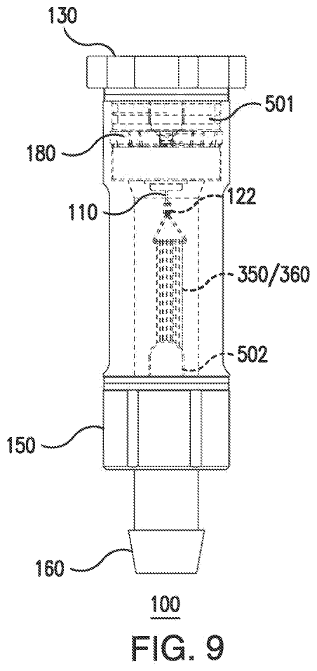

In preferred aspects, the sampling device has tamper-evident seals (see, for example, FIG. 9, showing seals 501 and 502) so that the user can operate the device correctly and be alerted to avoid using the device if the device has been tampered with (e.g., prematurely opened which could contaminate the sample and/or expose the user to a health hazard). In some aspects, the retractor can be enclosed in a wrap (e.g., a plastic shrink-wrap) to further prevent accidental retraction.

The hollow tube should be formed from a material that can be weldable or clamped/crimped to maintain an aseptic disconnection from the bioreactor system.

Illustrative suitable materials for the hollow tube include thermoplastic elastomers and silicone.

The cassette can be fabricated from any suitable rigid impervious material, including any impervious thermoplastic material, which is compatible with the fluid being processed. In a preferred aspect, the frame is a polymer, in some aspects, a transparent or translucent polymer, such as an acrylic, polypropylene, polystyrene, or a polycarbonated resin.

Aspects of the sampling device are suitable for use in a variety of cell culture and/or bioreactor systems, e.g., for use in commercially available fixed-bed bioreactor systems, for example, the iCELLis® Single-Use Fixed-Bed Bioreactor System, including the iCELLis® Nano System and the iCELLis® 500 System (Pall Corporation, Port Washington, NY, USA).

A variety of methods for assembling the sampling device in the bioreactor system are suitable.

For example, the cassette frame 310 is inserted and can be snapped into position into the fixed bed system 2000 (e.g., wherein the fingers 312A (if present) engage with the bottom plate 2002, or the cassette frame is sandwiched between the top and bottom plate 2003, 2002 or the cassette frame is friction-fit into the bottom), e.g., at the time of manufacture of the bioreactor system, and the bioreactor lid 2001 is sealed onto the system.

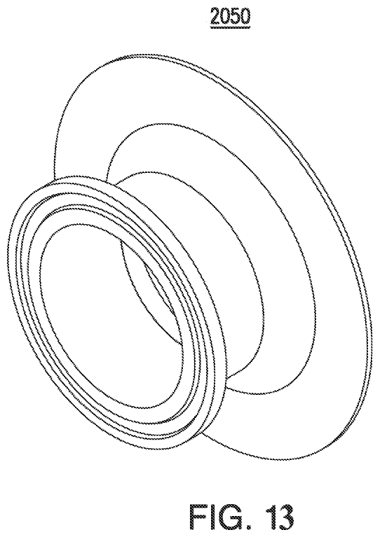

A bioreactor interface sanitary flange 2050 (see, FIGS. 1A-1I and 13) is welded or bolted into place on the lid 2001. O-rings are all put into place in the knob (136A, 136B in grooves 135A, 135B) and end cap (175 in groove 174). The retraction filament is cut to length.

The retraction filament 110 is fed through the aperture 131 in the knob 130 and the first stopper 121 is attached to the filament. The retraction filament is pulled back through the aperture 131 to seat the attached first stopper. The retraction filament is fed through the top hole 187B in the insert cylinder 180, and through the bottom hole 187A in the insert cylinder. The knob is inserted into position through the insert cylinder and the retaining ring 137 is put in place in groove 139A.

The retraction filament is fed through the retraction chamber 170 and the shoulder 183 of the insert cylinder is aligned with the cut-out 178 of the retraction chamber and the knob assembly with the insert cylinder is inserted with enough force that the insert cylinder arms 180B “snap” into place in the snap groove 179. The retraction filament is fed through the end cap 150 and the end cap is threaded on the end 172 of the retraction chamber. The retraction filament is fed through the tube 200 and the tube end 201 is attached to the first hollow connector 160 on the end of the retraction assembly 100.

The retraction filament is fed through the second stopper 122 and the stopper 122 is slid up the retraction filament several inches. The lower end of the retraction filament is fed through the aperture(s) 365 (at the end 361 of the sampling element in the aspect shown in FIGS. 5A-5C) and fed back on itself, leaving enough slack for the sides of the pleats of the sampling element to lay flush against each other. The retraction filament end is fed back through the attached second stopper 122 and crimped into place, leaving the sampling element attached to a loop in the retraction filament.

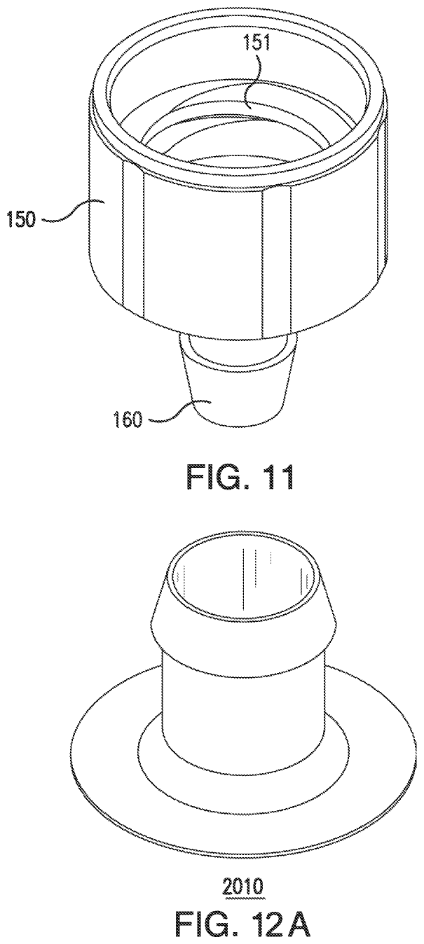

The sampling element 350 is fed through the top of the connector 2010 (shown as a hose barb; see, FIGS. 1A-1I and 12A), or connector 2010′ (also shown as a hose barb; see, FIGS. 1F-1G and 11B) on the bioreactor and the connector is inserted into the bottom end 202 of the tube 200. The sampling element is inserted (e.g., using a tweezers) into the fixed-bed system between the bottom plate 2002 and the upper plate 2003, or into the cassette frame 350 and pushed down until it contacts the support 321. In those aspects including the optional retainer, the sampling element is retained by the retainer (e.g., legs 311B). The connector is put in place on the sanitary flange 2050 on the bioreactor lid 2001 and secured with a clamp 2075 (such as a tri-clover clamp; see, FIGS. 14A-14B). The knob can be rotated to remove excess slack in the retraction filament. The tamper-evident seals 501 and 502 are affixed to the retraction assembly.

The following example further illustrates the invention but, of course, should not be construed as in any way limiting its scope.

### EXAMPLES

Example 1 shows cells adhere to a sampling element according to an aspect of the invention, wherein the sampling element is inserted into a fixed-bed bioreactor system.

A cassette with a sampling element as generally shown FIGS. 3A and 5A, is snapped into position into a fixed-bed bioreactor system (iCELLis® Nano System; Pall Corporation, Port Washington, NY, USA). The fixed-bed bioreactor system also has, in another portion of the system, loose macrocarriers (carrier strips), as used in the commercially available unmodified fixed-bed bioreactor system discussed in the following paragraph.

As a control, a commercially available unmodified fixed-bed bioreactor system including loose macrocarriers (carrier strips) (iCELLis® Nano System; Pall Corporation, Port Washington, NY, USA) is operated.

The sampling element is formed by 4 vertically aligned sheets of non-woven hydrophilized PET, wherein the total surface area of the sampling element available for cells to adhere to is approximately 56 cm². A monofilament is attached to the sampling element to allow retraction of the sampling element from the fixed-bed bioreactor system.

The fixed-bed reactor systems are operated for 3 days, and stopped. The sampling element is retracted, the carrier strips are removed, and both are analyzed.

Several cell culture experiments are performed to analyze how representative the cells captured by the sampling element are to the rest of the bioreactor (i.e., by analyzing the carrier strips) and to the control bioreactor (i.e., by analyzing the carrier strips). The seeding cell density, transfection cell density, and production phase times of the cell culture run are chosen to represent conditions that would yield minimal cell population density and viral vector titer (e.g., extreme, rather than optimum, conditions). These conditions include a 10,000-15,000 cells/cm² seeding density, 100,000-150,000 cells/cm² transfection cell population density, and a 2-day production phase. After the completion of the experiments, the bioreactor fixed bed carrier strips and the sampling element are compared using several methods.

The sampling elements and carrier strips from the bioreactor and from the control bioreactor are exposed to a crystal violet stain to allow imaging and observation of cell distribution, and the results show that the cells are evenly distributed across the surfaces of both the carrier strips and the sampling element.

The cell population density and viability are mapped across the sampling element and carrier strip surfaces. The results show that the average cell population density on the sampling element is within experimental uncertainty to that of the carrier strips and that the cell population density distribution on the sampling element corresponds with the crystal violet staining analysis. The results also show that the viability of the cells from the sampling element and the carrier strips are similar and greater than 90%.

The carrier strips and the sampling element are also imaged with fluorescent microscopy to verify that the cells had been transfected with green fluorescent protein. This shows that the cells on the sampling element and on the carrier strips are transfected. The cell population density collected by the sampling element is 51,000 to 162,000cells/cm².

Cells are removed from the sampling element and the various carrier strips, and seeded into fresh cell media-containing culture flasks, to compare their post-processing population doubling times. Similar post-processing population doubling times are observed in both the cells from the sampling element and from the carrier strips.

Example 2 shows cells adhere to a sampling element in a cassette according to an aspect of the invention, wherein the cassette is inserted into a fixed-bed bioreactor system.

A cassette with a sampling element as generally shown FIG. 3A is snapped into position into a fixed-bed bioreactor system (iCELLis® Nano System; Pall Corporation, Port Washington, NY, USA). The fixed-bed bioreactor system also has, in another portion of the system, loose macrocarriers (carrier strips), as used in the commercially available unmodified fixed-bed bioreactor system discussed in the following paragraph.

As a control, a commercially available unmodified fixed-bed bioreactor system including loose macrocarriers (carrier strips) (iCELLis® Nano System; Pall Corporation, Port Washington, NY, USA) is operated.

The sampling element is formed by 4 vertically aligned pleated sheets of non-woven hydrophilized PET, taking up 60% of the flow area in the cassette, wherein the total surface area of the sampling element available for cells to adhere to is approximately 4000cm². A monofilament is attached to the sampling element to allow retraction of the sampling element from the cassette.

The fixed-bed reactor systems are operated for 3 days, and stopped. The sampling element is retracted, the carrier strips are removed, and both are analyzed.

Several cell culture experiments are performed to analyze how representative the cells captured by the sampling element are to the rest of the bioreactor (i.e., by analyzing the carrier strips) and to the control bioreactor (i.e., by analyzing the carrier strips). The seeding cell density, transfection cell density, and production phase times of the cell culture run are chosen to represent conditions that would yield minimal cell population density and viral vector titer (e.g., extreme, rather than optimum, conditions). These conditions include a 10,000-15,000 cells/cm² seeding density, 100,000-150,000 cells/cm² transfection cell population density, and a 2-day production phase. After the completion of the experiments, the bioreactor fixed bed carrier strips and the sampling element are compared using several methods.

The sampling elements and carrier strips from the bioreactor and from the control bioreactor are exposed to a crystal violet stain to allow imaging and observation of cell distribution, and the results show that the cells are evenly distributed across the surfaces of both the carrier strips and the sampling element.

Small samples are cut from the sampling element and the various carrier strips so that the cell population density and viability can be mapped across the sampling element and carrier strip surfaces. The results show that the average cell population density on the sampling element is within experimental uncertainty to that of the carrier strips and that the cell population density distribution on the sampling element corresponds with the crystal violet staining analysis. The results also show that the viability of the cells from the sampling element and the carrier strips are similar and greater than 90%.

The various carrier strips and the sampling element are also imaged with fluorescent microscopy to verify that the cells had been transfected with green fluorescent protein. This shows that the cells on the sampling element and on the carrier strips are transfected. The total number of cells collected by the sampling element is 3.5 to 7.9×10⁸ cells, which exceeds the FDA minimum of 1×10⁸ cells for replication competent retrovirus testing.

Cells are removed from the sampling element and the various carrier strips, and seeded into fresh cell media-containing culture flasks, to compare their post-processing population doubling times. Similar post-processing population doubling times are observed in both the cells from the sampling element and from the carrier strips.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law.

Moreover, any combination of the above-described elements in all possible variations thereof is encompassed by the invention unless otherwise indicated herein or otherwise clearly contradicted by context.

1. A sampling device for use in fixed-bed cell culture bioreactor systems comprising:
A rotatable knob;
an end cap including a hollow connector;
a hollow retraction chamber having a first chamber end and a second chamber end, wherein the first chamber end is sealed against the rotatable knob, and the second chamber end is connected to, and in fluid communication with the end cap;
a hollow tube having a tube first end and a tube second end, the tube first end connected to, and in fluid communication with the hollow connector of the end cap, and the tube second end configured for connection to a lid of a fixed-bed cell culture bioreactor system;
a sampling element embedded inside the fixed-bed cell culture bioreactor system; and
a retraction filament that connects the rotatable knob to the sampling element,
wherein rotating the rotatable knob pulls the retraction filament and the sampling element in an upward direction through the hollow tube toward the rotatable knob and into the hollow retraction chamber.

2. The sampling device of claim 1, wherein the sampling element is porous and comprises a porous medium.

3. The sampling device of claim 1, wherein the porous sampling element comprises a pleated porous sampling element comprising a pleated porous medium.

4. The sampling device of claim 3, wherein the pleated porous sampling element comprises a continuous pleated porous medium.

5. The sampling device of claim 1, wherein the porous sampling element comprises a plurality of arms.

6. The sampling device of claim 1, further comprises a hollow insert cylinder interposed between the knob and the hollow retraction chamber.

7. The sampling device of claim 1, wherein rotating the rotatable knob pulls the retraction filament and the sampling element in an upward direction toward the rotatable knob and into the hollow retraction chamber until the sampling element is prevented from further upward movement and the retraction filament is broken.

8. The sampling device of claim 1, wherein the rotatable knob contains an aperture, wherein the retraction filament passes through the aperture, and a first stopper, attached to the filament, is seatable in the aperture to connect the retraction filament to the rotatable knob.

9. The sampling device of claim 1, further comprising a cassette, the cassette comprising:
a frame having an open top end; a bottom end; a longitudinal axis from the open top end to the bottom end; a right side wall; a rear wall; a left side wall; a front wall;
an open interior; and a horizontally arranged support between the top end and the bottom end;
wherein the sampling element is located in the open interior of the frame,
wherein the sampling element has an upper element end and a lower element end, and the aperture comprises horizontally arranged apertures passing through sampling element near the upper element end, the sampling element being supported at the lower element end by the horizontally arranged support, and
wherein the retraction filament passes through the horizontally arranged apertures.

10. The sampling device of claim 1, wherein the retraction filament contains a second stopper near the sampling element; wherein the retraction filament passes through the aperture of the second stopper, through the aperture or horizontally arranged apertures of the sampling element, and further passes back through the stopper and forms a loop.

11. The sampling device of claim 9, wherein the open top end of the cassette frame has a retainer comprising at least two opposing inwardly facing legs, and the right side wall and the left side wall each have one of the at least two inwardly facing legs, and the front wall and the rear wall each have one of two opposing notches; or
the rear wall and the front wall each have one of the at least two inwardly facing legs, and the right side wall and the left side wall each have one of the two opposing notches, and the sampling element is retained in the frame by the at least two inwardly facing legs.

12. The sampling device of claim wherein the bottom end of the frame of the cassette further comprises two or more fingers configured to engage with a bottom plate in the fixed-bed cell culture bioreactor system.

13. A system for use in a fixed-bed cell culture bioreactor system comprising:
the sampling device of claim 1;
a second hollow connector connectable to the tube second end;
a flange connectable to the second hollow connector; and
a clamp configured to clamp the tube second end to the second hollow connector and the second hollow connector to the flange.

14. A method for removing a cell sample from a fixed-bed bioreactor system, the method comprising rotating the rotatable knob of the sampling device of claim 1, to pull the retraction filament and the sampling element from a fixed-bed cell culture bioreactor system into the hollow retraction chamber until the sampling element is prevented from further upward movement and the retraction filament is broken; and,
while the hollow tube first end remains connected to, and in fluid communication with, the hollow connector of the retraction assembly, aseptically disconnecting the hollow tube from a lid of the fixed-bed cell culture bioreactor system.

15. The method of claim 14, wherein the retraction filament is broken when the second stopper prevents further movement of the filament during rotation of the rotatable knob.
